(19) United States
(12) Patent Application Publication
(10) Pub. No.: US 2026/0076767 A1
(43) Pub. Date: Mar. 19, 2026

(54) MEDICAL EQUIPMENT MOUNTING SYSTEM HAVING ADAPTIVE MOUNT

(71) Applicant: SLD Technology, Inc., Portland, OR (US)

(72) Inventors: Mikah BARNETT, Clackamas, OR (US); Matthew YUNG, Sandy, OR (US); Jason WILKINSON, Battleground, WA (US)

(73) Assignee: SLD Technology, Inc., Portland, OR (US)

(21) Appl. No.: 19/310,570

(22) Filed: Aug. 26, 2025

Related U.S. Application Data
(60) Provisional application No. 63/687,188, filed on Aug. 26, 2024.

(51) Int. Cl. A61B 90/50

(52) U.S. Cl. CPC A61B 90/50

(57) ABSTRACT

Systems and methods for mounting medical equipment in an adaptable manner are provided. In one example, a mounting system include a mount having a frame defined by opposing walls and a plurality of support members located at corner regions of the frame. The plurality of support members includes one or more features configured to align a plurality of different types of mounting plates to the frame.

### CROSS-REFERENCE TO RELATED APPLICATIONS

This application claims the benefit of and priority to U.S. Provisional Application No. 63/687,188, filed Aug. 26, 2024. The disclosure of the prior application is hereby incorporated by reference herein in its entirety.

### BACKGROUND

The statements in this section merely provide background information related to the present disclosure and may not constitute prior art.

Hospital operating rooms typically include surgical equipment and one or more lights that are mounted over a surgical site. The surgical equipment may be movable in relation to a surgical site target zone (e.g., equipment on a movable arm mounted to the ceiling).

In many mounting systems, the mounting arrangement for the surgical equipment is customized with a fixed arrangement for the mounting locations, mounting structures, mounting configuration, etc. Accordingly, if different equipment is to be installed in different operating rooms (e.g., equipment from different manufacturers), a new mounting arrangement may be needed, which can result in having to redesign a portion of, or even the entire mounting arrangement. Alternatively, costly changes may have to be made to accommodate the different equipment.

### SUMMARY

This section provides a general summary of the disclosure and is not a comprehensive disclosure of its full scope or all of its features.

One or more techniques and systems are described herein for mounting medical equipment in a configurable manner. In one example, a medical equipment mounting system includes an adaptive outboard mount. For example, an adaptive plate outboard mount is provided that allows for coupling different mounting plates to a mounting system.

In one or more embodiments, a medical equipment mounting system is provided. In one example, the medical equipment mounting system includes a mount having a frame defined by opposing walls and a plurality of support members located at corner regions of the frame. The plurality of support members includes one or more features configured to align a plurality of different types of mounting plates to the frame.

In one or more embodiments, systems for mounting medical equipment in an adaptable manner are provided. In one example, a mounting system include a mount having a frame defined by opposing walls and a plurality of support members located at corner regions of the frame. The plurality of support members includes one or more features configured to align a plurality of different types of mounting plates to the frame.

Further areas of applicability will become apparent from the description provided herein. It should be understood that the description and specific examples are intended for purposes of illustration only and are not intended to limit the scope of the present disclosure.

### DRAWINGS

In order that the disclosure may be well understood, there will now be described various forms thereof, given by way of example, reference being made to the accompanying drawings, in which:

FIG. 1 illustrates a lateral view of an operating room according to an example.

FIG. 2 illustrates a perspective bottom view of a supply air array according to one or more examples.

FIG. 3 illustrates a perspective view of a mounting system according to various examples.

FIG. 4 illustrates a top perspective view of a mount according to various examples.

FIG. 5 illustrates a bottom perspective view of the mount of FIG. 4 showing a plate according to various examples.

FIG. 6 illustrates a side elevation view of the mount of FIG. 4 according to various examples.

FIG. 7 illustrates a top perspective view of the mount of FIG. 4 according to various examples with a wall removed.

FIG. 8 illustrates another top perspective view of the mount of FIG. 4 according to various examples with a wall removed.

FIG. 9 is an enlarged view of a corner portion of the mount of FIG. 4 according to various examples.

FIG. 10 is a perspective view of a plate according to various examples.

FIG. 11 illustrates a top perspective view of the mount of FIG. 4 according to various examples with a wall in phantom.

FIG. 12 illustrates a perspective view of a portion of the mount of Figure according to various examples with a wall in phantom.

FIG. 13 illustrates a perspective view of a portion of the mount of Figure according to various examples with a wall in phantom.

FIG. 14 illustrates a perspective view of a portion of the mount of Figure according to various examples with a wall in phantom.

FIG. 15 illustrates a top perspective view of the mount of FIG. 4 showing a different plate according to various examples.

FIG. 16 illustrates a top perspective view of the mount of FIG. 4 according to various examples without a plate.

FIG. 17 is a perspective view of a corner portion of the mount of Figure according to various examples.

FIG. 18 illustrates a perspective view of a portion of the mount of Figure according to various examples with a wall in phantom.

FIG. 19 illustrates a top perspective view of the mount of FIG. 4 according to various examples without a plate.

FIG. 20 is a perspective view of a portion of the mount of FIG. 4 according to various examples.

FIG. 21 is a side cutaway view of a portion of the mount of FIG. 4 according to various examples.

FIG. 22 illustrates a bottom perspective view of the mount of FIG. 4 showing the different plate according to various examples.

The drawings described herein are for illustration purposes only and are not intended to limit the scope of the present disclosure in any way.

### DETAILED DESCRIPTION

The following description is merely exemplary in nature and is not intended to limit the present disclosure, application, or uses. It should be understood that throughout the drawings, corresponding reference numerals indicate like or corresponding parts and features.

The claimed subject matter is now described with reference to the drawings, wherein like reference numerals are generally used to refer to like elements throughout. In the following description, for purposes of explanation, numerous specific details are set forth in order to provide a thorough understanding of the claimed subject matter. It may be evident, however, that the claimed subject matter may be practiced without these specific details. In other instances, structures and devices are shown in block diagram form in order to facilitate describing the claimed subject matter.

The methods and systems disclosed herein, for example, may be suitable for use in different mounting applications, such as with different mounting plates, such as in different medical or clinical settings. That is, the herein disclosed examples can be implemented in combination with equipment configurations or assemblies other than for particular applications, such as other than for particular operating room arrangements (e.g., surgical suites).

Various examples provide an outboard mount (e.g., externally mountable component or member) for a mounting system or arrangement for mounting equipment, such as surgical equipment within an operating room. However, it should be appreciated that the mount in some examples may be varied, such as to be an inboard mount or other type of mount. For example, in various embodiments, an adaptive mount (e.g., internally mountable component or member) is provided that allows for mounting of different equipment, which may have, for example, different mounting plates to allow for a more flexible truss arrangement for the mounting system. One or more herein described embodiments allow for easy adaptable coupling of surgical equipment (and other devices or members) to different locations within the operating room (e.g., to different locations of a supply air array 106), as well as flexibility to couple different components with different mounting plates using the mounting system, which in some examples reduces scheduling or manufacturing delays or issues with having different mounting plates.

In some examples, the mounting arrangement also allows for easy mounting of different equipment with different mounting configurations, such as having different mounting requirements, different mounting plates, etc. That is, the adaptable mount allows adjustability in multiple dimensions to provide different configurations of mounting arrangements. Additionally, one or more examples also allow for providing air supply at different locations of the supply air array 106. As such, one or more examples are adaptable to many different settings, room configurations, equipment configurations, etc.

For example, interchangeable mounting of access panels, light troffers and boom mounts for surgical equipment is facilitated and easily configurable during installation and setup, as well when changes are desired or needed. That is, one or more mechanisms or features of the adaptable mount arrangement allows for flexibility in mounting different equipment at different desired or required locations of a fixed truss structure. As such, one or more examples provide a flexible mounting system that allows for the easy configurable design and redesign (or reconfiguration) of the operating room or medical room (during installation or at a later time) without a change in the mounting structure.

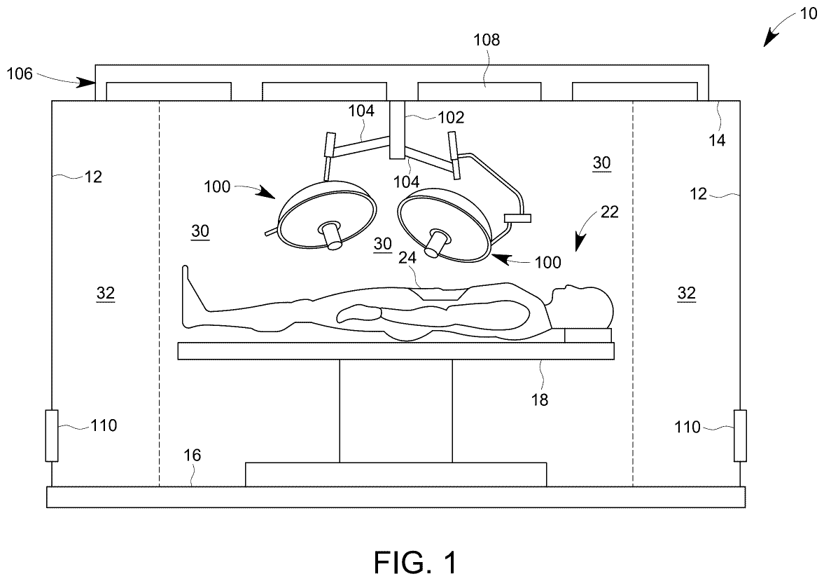

FIG. 1 illustrates a lateral view of an operating room 10, according to one example. The operating room 10 is defined by walls 12, a ceiling 14, and a floor 16. An operating table 18 is supported on the floor 16. The operating table 18 may include a support bed 20 that is configured to support a patient 22. A surgical site 24 may be located on the patient 22.

Surgical equipment, which in the illustrated embodiment is a surgical light system having surgical light assemblies 100 suspended from the ceiling 14 above the operating table 18, which may define a sterile field 30. A support beam 102 extends downwardly from the ceiling 14 and is coupled to a mounting system and providing adaptable mounting system as described in more detail herein. The adaptable mounting system in various examples provides an adaptable perimeter (outboard) truss mounting arrangement that allows for adjustability in mounting different types of medical equipment (that may have different mounting arrangements) around a perimeter of the sterile field 30. Accordingly, different equipment mounts (e.g., different mounting plates) are easily mountable at different locations, such as outside of the sterile field 30. In the illustrated example, this adaptability allows for changing the types of equipment mount(s) (e.g., different mounting plates) that support, for example, one or more boom arms 104 that extend from the support beam 102 into the sterile field 30 (or other equipment). As shown in FIG. 1, two surgical light assemblies 100 may be coupled to two separate and distinct boom arms 104 mounted to different locations on the mounting system, which may be performed in an adaptable and configurable manner during installation or thereafter during a reconfiguration or installation of different and/or additional equipment. Thus, more or less surgical light assemblies 100 or other medical equipment than shown may be used and mounted according to one or more examples described herein. The surgical light system is shown only for illustrative purpose and different or additional surgical equipment may be mounted to and suspended from the ceiling 14.

A supply air array 106 (also referred to as an air frame system) is secured to the ceiling 14 and configurable with the mounting system in one or more embodiments as described in more detail herein. The supply air array 106 may be provided as described, for example, in U.S. Pat. Nos. 9,903,115, 10,405,952, and 11,259,893. By positioning the mounting plates 42 outside of (along the perimeter) of the supply air array 106, laminar airflow is created directly to the surgical target zone without turbulence that could otherwise be caused by having the mounting plates 42 within the supply air array 106 or within the sterile field 30. However, the mounting plates 42 may be mounted at other locations. As such, in various embodiments, a specific plate does not have to be welded or otherwise permanently coupled to particular mounting structure locations.

The mounting and positioning of different mounting plates 42 are also easily adaptable (which in some embodiments provides a more universal mounting arrangement) to further facilitate a frame structure that allows for proper airflow within the surgical target zone even when surgical equipment is positioned above the surgical target zone. In various examples, low pressure zones that could cause air turbulence underneath the surgical equipment are reduced or eliminated. As such, in various examples, because the turbulent airflow is reduced or eliminated, various contaminants generated through a surgical procedure are not circulated within the surgical environment.

One or more examples provide an airframe structure that is configured to channel air, which may be filtered, sterilized or purified, to the surgical target zone with minimal or no air turbulence underneath the surgical equipment and that is mounted as described in more detail herein. Various examples thereby provide an integrated and modular arrangement that is easily adaptable to effectively deliver airflow directly to the surgical target zone while allowing adjustability in the mounting of equipment along the perimeter of the airframe structure using different types of mounts and equipment.

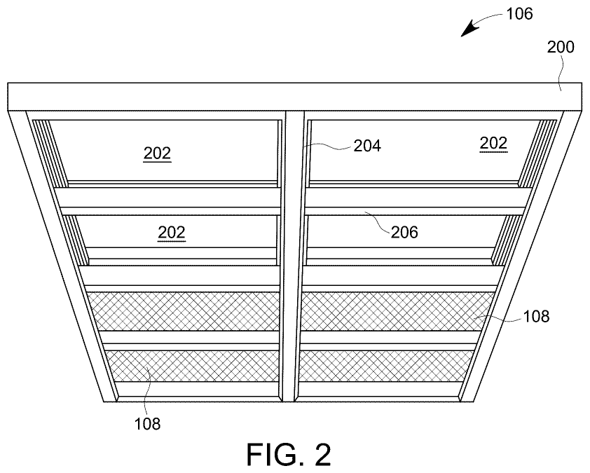

FIG. 2 illustrates a perspective bottom view of the supply air array 106, according to one example. The supply air array 106 in the illustrated example includes a frame 200 (a portion of which is shown and that forms part of a truss structure in various examples) having a plurality of openings 202 defined therein by cross-members 204 and 206. It should be noted that although the illustrated example shows a 2 cell x 4 cell array, the supply air array 106 may be sized differently (and pre-defined as described in more detail herein), including having a single opening 202. Additionally, the openings 202 may be sized and shaped differently than illustrated, for example, based on design requirements or constraints. In the example shown in FIG. 2, the front four openings 202 are illustrated with nothing therein and the back four opening 202 illustrate air diffusers 108 coupled within the openings 202. As can be seen, the air diffusers 108 are coupled with the openings 202 such that the air diffusers 108 are recessed within the openings 202 in this example. The frame 200 in various examples defines the supply air array 106 and has a plurality of removable portions, wherein the plurality of removable portions are positioned at airflow inlets of the supply air array and covering the airflow inlets. The frame 200 is also an example or a structure to which the mounting system having adaptable mounts described herein can be used to provide different perimeter truss mounting arrangements using different mounting plates (also referred to as mount plates) that allows for adaptability in mounting equipment at many different locations that are easily configurable and that reduces wait times for installing specific plates.

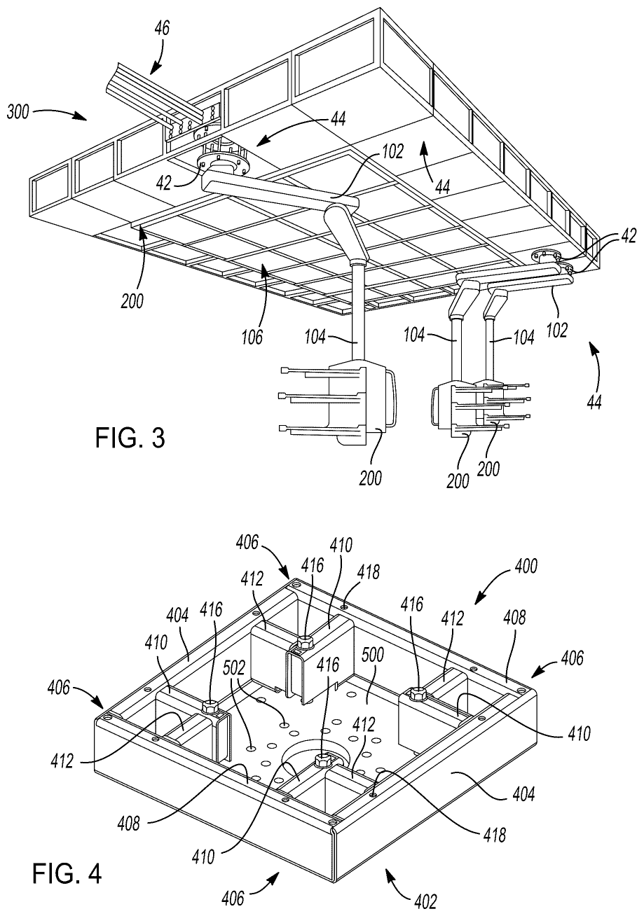

FIG. 3 illustrates a mounting system 300 and shows mounting plates 42 mounted to different mounting locations 44 defined within the truss structure of the mounting system 300. The mounting system 300 allows for interchangeable mounting of different mounting plates 42 (e.g., having different types, sizes, manufacturers, etc.) to any location along the perimeter of the frame 200 to thereby define plural mounting locations 44 that are easily adaptable with the mounting system 300 as described in more detail herein. The frame 200 defines the supply air array 106 that can have the adaptable mounting locations 44 and optionally or alternatively have a plurality of removable portions. For example, as described in more detail herein, the plurality of removable portions are positioned at airflow inlets of the supply air array 106 to allow and cover the airflow inlets as described in more detail herein. This configuration provides an adjustable air supply path to the supply air array 106.

Thus, the mounting locations 44 are movably provided along a perimeter of the supply air array 106 and are easily configurable and reconfigurable for use with different mounting plates. Accordingly, for example, the mounting system 300 allows for easy adjustment of the mounting locations 44, as well as the air supply locations, using different mounting arrangements. It should be noted that the different mounting locations allow for mounting the mounting plates 42 or, for example, other equipment mounts, thereon. However, the mounting locations 44 also provide interchangeable mounting of different components thereto, such as access panels or light troffers, among other components. For example, the mounting system 300 may be provided on one, two, three, or all four sides of the supply air array 106. As can be seen, the mounting locations 44 may be configured differently, such as to define different mounting types, such as different configurations of mounting plates 42 that include different mounting patterns of bore locations for securing thereto different components (e.g., bolt connection), such as from different manufacturers. One or more examples allow for mounting of medical equipment using different vendor specific or manufacturer specific mounting plates.

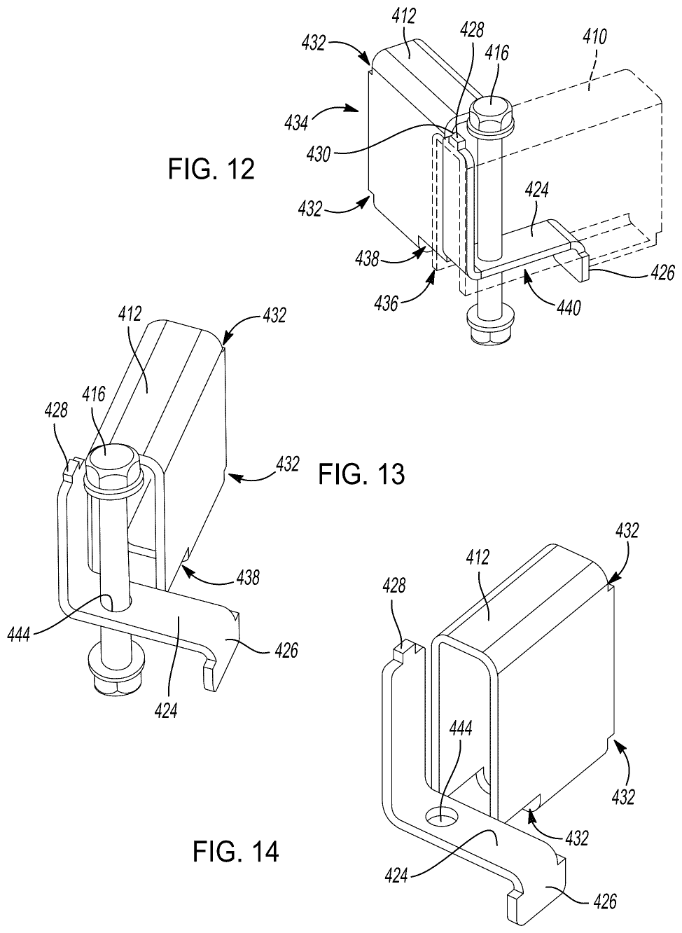

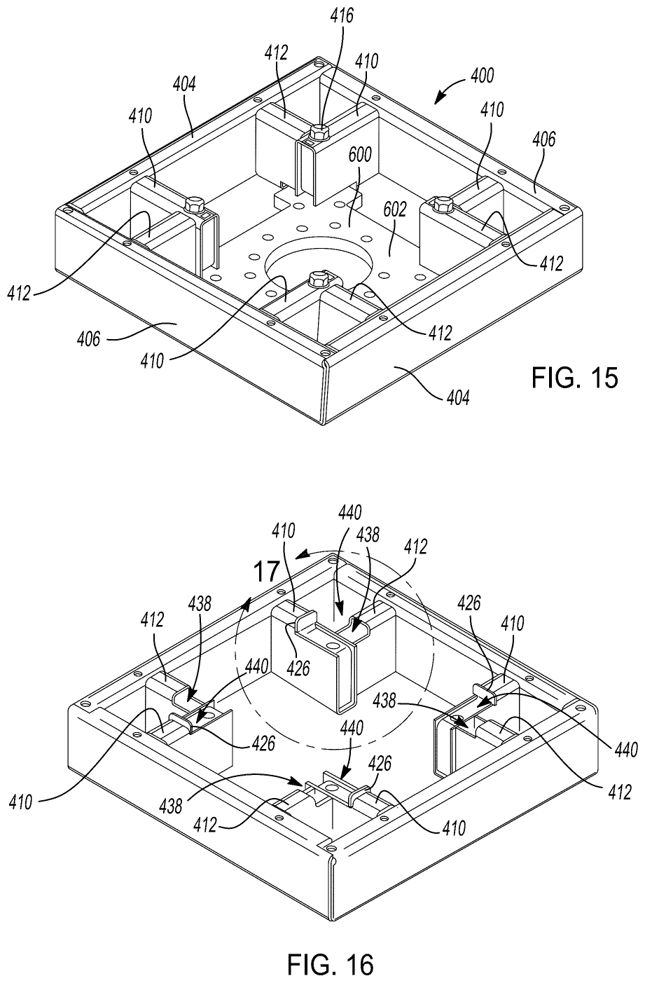

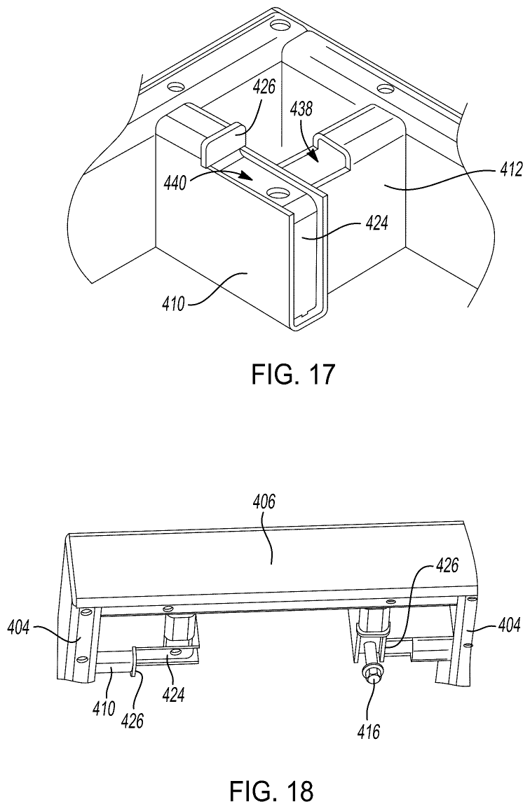

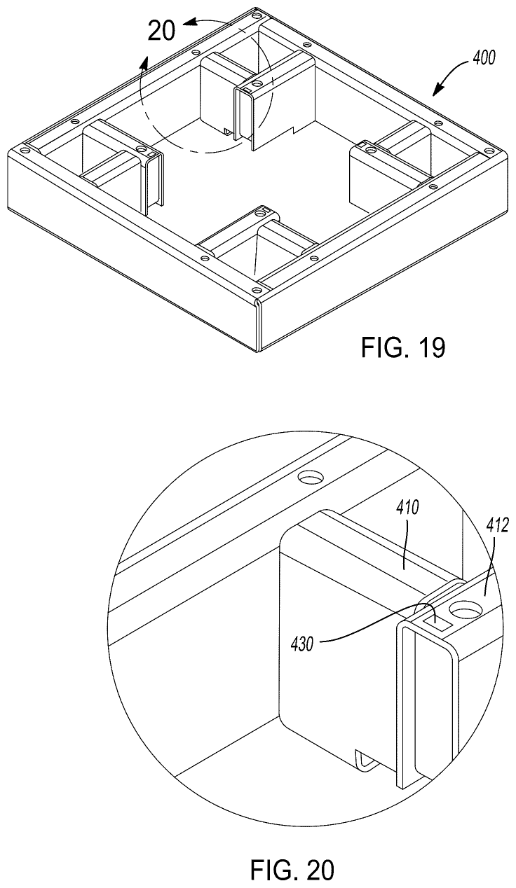

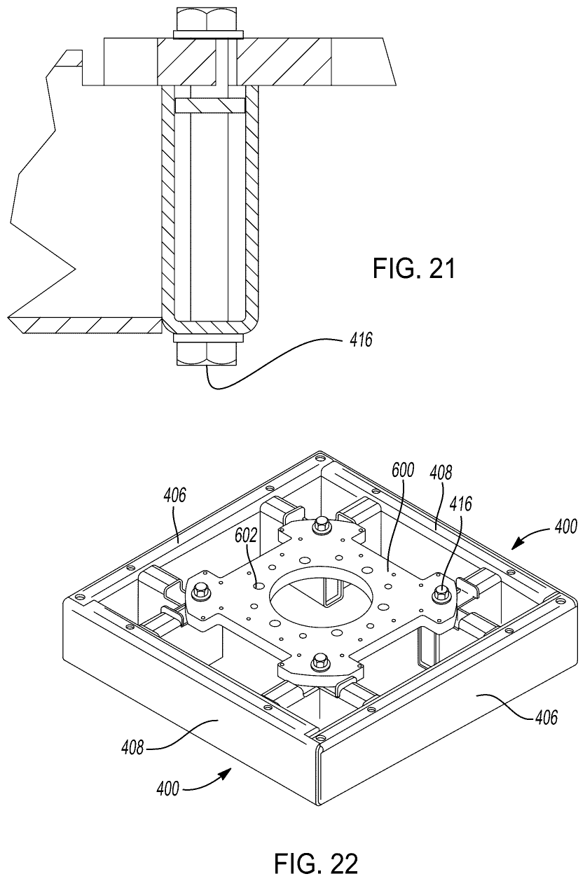

FIGS. 4-22 illustrate a mount 400, configured as an adaptive plate outboard mount arrangement that allows for mounting different types of plates thereto as described in more detail herein. As can be seen, FIGS. 4-14 illustrate the mount 400 having a plate 500 coupled thereto, and FIGS. 15-22 illustrate the mount 400 having a plate 600 coupled thereto. It should be appreciated that the plates 500, 600 (which may be embodied as or represent one or more of the mounting plates 42) are merely shown for example and the mount 400 is capable of receiving and/or having mounted thereto different types of plates than are shown.

The mount 400 allows for adaptable coupling of the plates 500, 600 to the frame 200 to thereby define multiple mounting locations 44. That is, the mount 400 allows for variable positioning of the mounting plates 500, 600 or other equipment mounts along the outside or the perimeter of the frame 200, or at other locations. In some examples, the frame 200 forms part of the supply air array 106 as described in more detail herein. It should be noted that any number of mounts 400 may be coupled to the frame 200, allowing for mounting of additional or fewer mounting plates 500, 600. As described in more detail herein, different types of mounting plates can be coupled to the frame 200, as well as other types of equipment mounts, such as for a patient chair lift or support for other medical devices or equipment.

As can be see, the mount 400 includes a mount frame 402 that includes a pair of opposing walls 404 and a pair of opposing walls 408. In one or more examples, the walls 404 and the walls 408 have a complementary configuration to allow for providing an integrated mounting structure as the mount frame 402. That is, the walls 404, 408 are coupled together (e.g., welded together) to form an outboard mount frame structure to which the mounting plates 500, 600, as well as other mounting plates, can be mounted. It should be noted that the walls 404, 408 may have the same or different dimensions, such as having the same or different length, height, and/or width.

In the illustrated example, the mount frame 402 is supported and/or reinforced at each corner 406 by a pair of support walls 410, 412 arranged in a perpendicular configuration. In some examples, the support walls 410, 412 are coupled together (e.g., welded together) and each is coupled to a different wall 404, 408. The support walls 410, 412 form support portions in addition to defining mounting locations for the mounting plates 500, 600. For example, the walls 410 include openings 414 therethrough for receiving a fastener, illustrated as a bolt 416, for coupling the mounting plate 500, 600 to the mount 400. As can be seen, the bolt 416 passes through the wall 410 and then through openings 418 of the mounting plate 500, 600 to allow for the mounting plate 500, 600 to be secured to the mount 400.

In the mounted position, the mounting plates 500, 600 allow for coupling of different medical equipment thereto. For example, mounting openings 502, 602 define a pattern of openings for the specific plate type that allows for mounting thereto of medical equipment having a complementary mounting arrangement (e.g., mounting of medical equipment from a particular manufacturer or vendor that is specific to the mounting plates 500, 600). It should be appreciated that any configuration of mounting openings 502, 602 may be provided.

The mount 400 further includes openings 418 through the walls 404, 408 that allows for the mount 400 to be coupled to another structure and/or to other mounting plates. For example, the openings 418 allow for the mount 400 to be coupled to a support structure, such as a truss. It should be noted that additional or fewer openings 418 may be provided and arranged and/or spaced differently than shown.

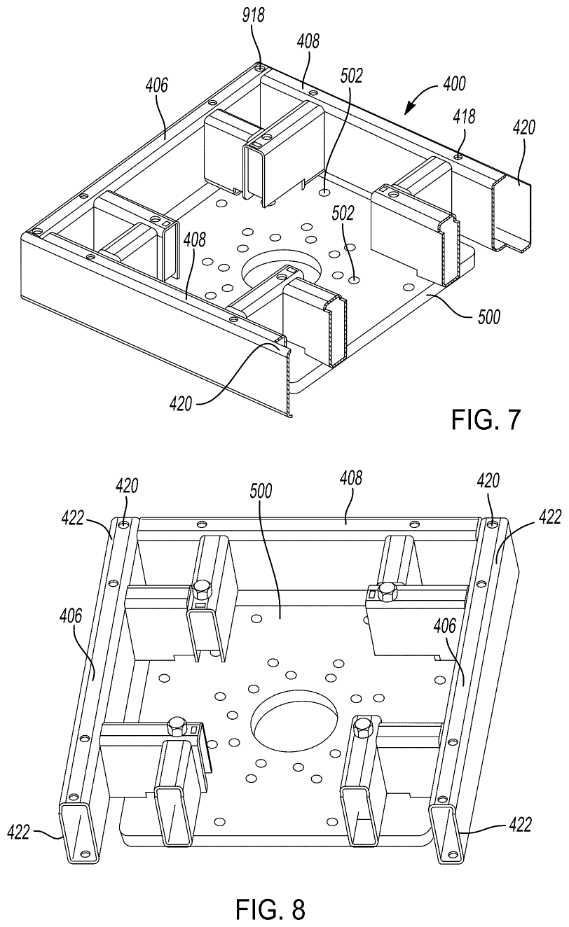

As can be seen for example in FIGS. 7 and 8, the walls 408 include extended portions 420, which may be configured as flaps or extensions that abut ends 422 of the walls 408 when coupled together to form the frame 402 of the mount 400. That is, the extended portions 420 of opposing walls are configured to receive therebetween the ends 422 of the walls 408 that are coupled thereto (e.g., welded together). The extended portions 420 generally define an outer wall extension that extends from the main tubular structure of the walls 408 that includes inner and outer wall portions with a hollow interior defined therebetween.

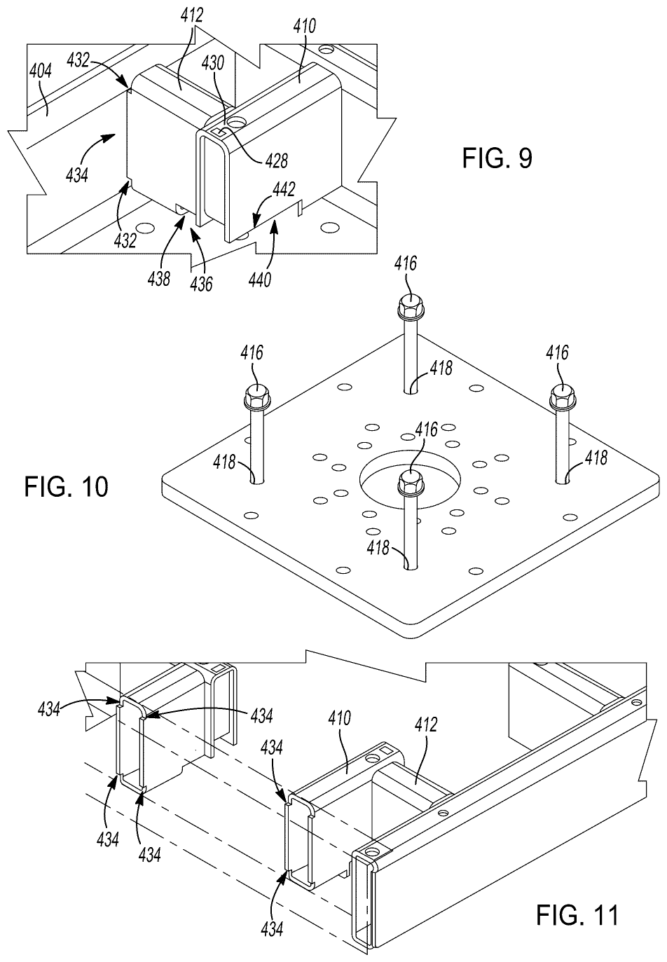

The support walls 410, 412 also include a complementary configuration to facilitate coupling together, such as shown in FIGS. 9 and 12-14. In one or more examples, a coupling member 424 facilitates alignment and/or coupling of the support walls 410, 412. That is, the coupling member 424 is generally L-shaped with a tab 428 at a top end thereof that is configured to be received within an opening 430 (e.g., slot) of the wall 410. That is, the tab 428 extends into the opening 430 of the wall 410 to facilitate aligning (e.g., locates and aligns) and securing the wall 410 to the wall 412. The coupling member 424 also includes an extension 426 at a lower end that is configured to facilitate aligning and/or maintaining the position of the mounting plates 500, 600 to the mount 400 as described in more detail herein. It should be noted that while the tab 428 is shown as being narrower than the width of the coupling member 424 and the extension 426 is shown being wider than the coupling member 424, other configurations are contemplated (e.g., different length, widths, thicknesses, etc.). As can be seen, the coupling member 424 is integrated within the wall 410 in the illustrated example and also includes an opening 444 therethrough for receiving the bolt 416. It should be noted that a portion of the coupling member 424 can be coupled to (e.g., welded to) one or more of the walls 410, 412.

In one or more embodiments, the support wall 412 includes cutouts 432 at an end 434 opposite an end 436 (adjacent to the coupling member 424). The cutouts 432 are provided at a top and bottom of the end 434 and define engagement portions or shoulders for engagement with the wall 404. That is, the cutouts 432 are configured to abut against an interior or inner portion of a top and a bottom of the wall 404. In particular, edges of the top and both of the wall 404 fit within the cutouts 432 when the support wall 412 is arranged and coupled to the wall 404. As such, the cutouts 432 facilitate alignment and/or positioning of the support wall 412 with the wall 404. The support wall 410 has cutouts 440 similar to the cutouts 432 to facilitate alignment and/or positioning of the support wall 410 with the wall 408.

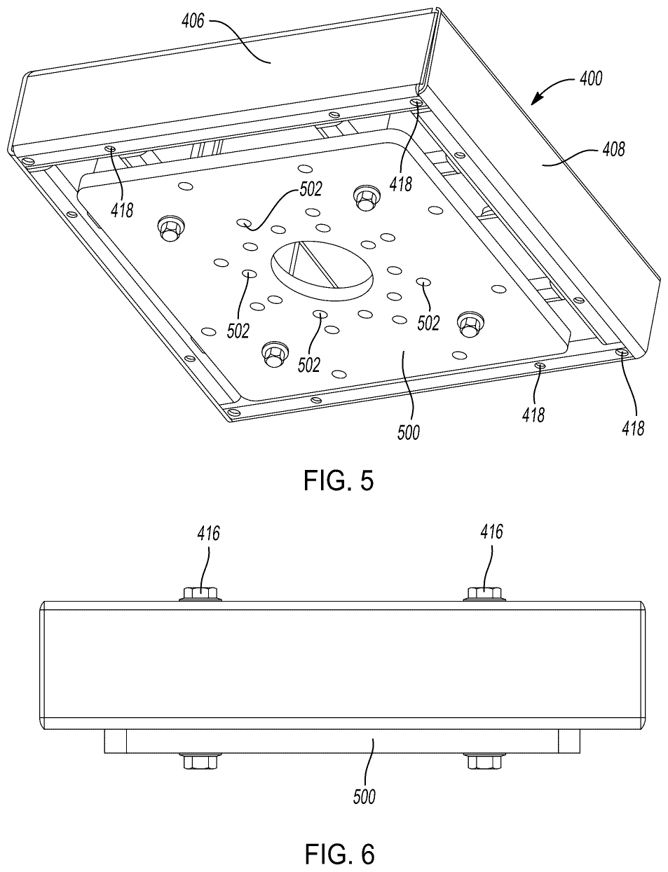

The support walls 410, 412 also include alignment features and/or supports on a bottom thereof. As can be seen, the support wall 412 includes a cutout 438 configured to be complementary to and facilitate alignment of the mounting plates 500, 600 to the mount 400. That is, the cutout 438 is sized and shaped to receive a portion of the mounting plate 500, 600 (e.g., an edge of the mounting plate 500, 600) therein or thereon. That is, the cutout 438 is formed by a bottom of the coupling member 424 and together with the extension 426 of the coupling member 424 facilitates alignment of the mounting plate 500, 600 in the corner region of the mount 400. As can be seen, the extension 426 extends below the bottom of the wall 410 with the bottom of the coupling member 424 extending along a narrower portion 442 of the wall 410 to define a cutout 440. This complementary arrangement of the cutouts also facilitate alignment and mounting of different types of mounting plates to the mount 400, for example, as can be seen in FIG. 5 with respect to the mounting plate 500 and FIG. 22 with respect to mounting plate 600.

Thus, the mount 400 is configured to receive different mounting plates to allow mounting of different equipment, such as from different manufacturers or vendors, thereto. In various examples, the configuration of the mount including the complementary features and cutouts allows for adaptability to mount different types of mounting plates, such as the mountings plates 500, 600 to the mount 400 using the bolts 416, which are illustrated as mounting in the corners of the mounting plates 500, 600. The complementary features and cutouts of the mount 400 accommodate variances in the configuration of different mounting plates, such as mounting plates having different dimensions, sizes, shapes, etc. For example, the mounting plates 500, 600 illustrate different mounting configurations that may be used and that allows for mounting of different medical equipment using the mount 400. In some examples, the mount 400 accommodates different mounting plates with the herein described features along with a rigidity to prevent the mounting plates from rotating when coupled to the mount 400. With the mount 400, field installation is made more adaptable to different mounting plate arrangements.

The mount 400 allows for mounting thereto of different mounting plates that may have different rotated configurations (e.g., plates having install or mounting configurations that are mounted in positions that are forty-five degrees rotated from one plate to another). In some examples, as described in more detail herein, support is provided by tube structures in the corner instead of crossing tubes. In some examples, instead of welding the plate to the drop tubing, the plate is bolted to the corner tubes as described in more detail herein. In various examples, the mount 400 is also configured to mount (e.g., bolt) to, for example, the airframe or other support structure to thereby allow receiving various different equipment mounting plates. In various examples, a universal adaptable mounting solution is thereby provided.

Thus, in one or more embodiments, a medical equipment mounting system includes a mount having a frame defined by opposing walls and a plurality of support members located at corner regions of the frame. The plurality of support members includes one or more features configured to align a plurality of different types of mounting plates to the frame. Each of the plurality of support members can include a first support wall and a second support wall arranged in a perpendicular configuration. The first support wall can include a coupling member having an alignment tab configured to be received within an alignment slot of the second support wall. The coupling member can further include a lower extension configured to facilitate alignment of the plurality of different types of mounting plates. The one or more features can include plate alignment cutouts formed in the plurality of support members, the plate alignment cutouts being sized and shaped to receive portions of the plurality of different types of mounting plates. The opposing walls can include extended portions configured to receive wall ends of adjacent opposing walls when the frame is assembled. A supply air array having a plurality of openings can be provided, wherein the mount is coupled to the supply air array along a perimeter thereof.

In one or more embodiments, a mount for medical equipment includes a frame having opposing walls that define a perimeter and a plurality of support walls positioned at corner regions of the frame, wherein each support wall includes a coupling member having an alignment tab configured to engage with an alignment slot of an adjacent support wall. A plurality of cutouts are formed in the support walls, wherein the cutouts are configured to receive and align different mounting plates to the mount. The coupling member can further include a lower extension that extends below a bottom surface of the support wall. The lower extension can be configured to facilitate alignment of the different mounting plates by providing a positioning reference. The plurality of cutouts can include plate alignment cutouts formed at bottom portions of the support walls, wherein the plate alignment cutouts are sized to receive edge portions of the different mounting plates. The plurality of cutouts can further include support wall cutouts that define engagement portions configured to abut against interior portions of the opposing walls. The opposing walls can include extended portions at ends thereof, the extended portions configured to receive wall ends of adjacent opposing walls when the frame is assembled. Each support wall can include mounting openings configured to receive fasteners for securing the different mounting plates to the mount.

In one or more embodiments, a medical equipment mounting system for an operating room includes a supply air array having a frame with a plurality of openings and an adaptive mount coupled to the supply air array, wherein the adaptive mount has a plurality of support members at corner regions, and wherein the support members include alignment features configured to accommodate mounting plates from different manufacturers. Each of the plurality of support members can include a first support wall and a second support wall arranged in a perpendicular configuration at each corner region. The first support wall can include a coupling member having an alignment tab configured to be received within an alignment slot of the second support wall. The coupling member can further include a lower extension that extends below a bottom surface of the first support wall to facilitate positioning of the mounting plates. The alignment features can include plate alignment cutouts formed in the support members, the plate alignment cutouts being sized and shaped to receive edge portions of mounting plates having different dimensions. The support members can further include support wall cutouts that define engagement portions configured to abut against interior portions of the supply air array frame when the adaptive mount is coupled thereto.

In one or more embodiments, a mount includes a frame defined by opposing walls and a plurality of support members located at corner regions of the frame, wherein the plurality of support members include one or more features configured to align a plurality of different types of mounting plates to the frame. The one or more features can includes one or more complementary cutouts. The one or more features can include one or more tabs. The one or more features can include one or more extensions. The plurality of support members can include perpendicularly oriented walls, and the one or more features can include one or more L-shaped coupling members within one or more of the perpendicularly oriented walls. The plurality of support members can be configured to mount the plurality of different types of mounting plates to the frame without welding. The plurality of support members can be configured to mount the plurality of different types of mounting plates to the frame using a plurality of bolts.

While various spatial and directional terms, including but not limited to top, bottom, lower, mid, lateral, horizontal, vertical, front and the like are used to describe the present disclosure, it is understood that such terms are merely used with respect to the orientations shown in the drawings. The orientations can be inverted, rotated, or otherwise changed, such that an upper portion is a lower portion, and vice versa, horizontal becomes vertical, and the like.

The word “exemplary” is used herein to mean serving as an example, instance or illustration. Any aspect or design described herein as “exemplary” is not necessarily to be construed as advantageous over other aspects or designs. Rather, use of the word exemplary is intended to present concepts in a concrete fashion. As used in this application, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Further, at least one of A and B and/or the like generally means A or B or both A and B. In addition, the articles “a” and “an” as used in this application and the appended claims may generally be construed to mean “one or more” unless specified otherwise or clear from context to be directed to a singular form.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims. Of course, those skilled in the art will recognize many modifications may be made to this configuration without departing from the scope or spirit of the claimed subject matter.

As used herein, a structure, limitation, or element that is “configured to” perform a task or operation is particularly structurally formed, constructed, or adapted in a manner corresponding to the task or operation. For purposes of clarity and the avoidance of doubt, an object that is merely capable of being modified to perform the task or operation is not “configured to” perform the task or operation as used herein.

Any range or value given herein can be extended or altered without losing the effect sought, as will be apparent to the skilled person.

Also, although the disclosure has been shown and described with respect to one or more implementations, equivalent alterations and modifications will occur to others skilled in the art based upon a reading and understanding of this specification and the annexed drawings. The disclosure includes all such modifications and alterations and is limited only by the scope of the following claims. In particular regard to the various functions performed by the above described components (e.g., elements, resources, etc.), the terms used to describe such components are intended to correspond, unless otherwise indicated, to any component which performs the specified function of the described component (e.g., that is functionally equivalent), even though not structurally equivalent to the disclosed structure which performs the function in the herein illustrated exemplary implementations of the disclosure.

In addition, while a particular feature of the disclosure may have been disclosed with respect to only one of several implementations, such feature may be combined with one or more other features of the other implementations as may be desired and advantageous for any given or particular application. Furthermore, to the extent that the terms “includes,” “having,” “has,” “with,” or variants thereof are used in either the detailed description or the claims, such terms are intended to be inclusive in a manner similar to the term “comprising.”

The implementations have been described, hereinabove. It will be apparent to those skilled in the art that the above methods and apparatuses may incorporate changes and modifications without departing from the general scope of this invention. It is intended to include all such modifications and alterations in so far as they come within the scope of the appended claims or the equivalents thereof.

The description of the disclosure is merely exemplary in nature and, thus, variations that do not depart from the substance of the disclosure are intended to be within the scope of the disclosure. Such variations are not to be regarded as a departure from the spirit and scope of the disclosure.

What is claimed is:

1. A medical equipment mounting system, comprising:
a mount having a frame defined by opposing walls and a plurality of support members located at corner regions of the frame, wherein the plurality of support members includes one or more features configured to align a plurality of different types of mounting plates to the frame.

2. The medical equipment mounting system of claim 1, wherein each of the plurality of support members comprises a first support wall and a second support wall arranged in a perpendicular configuration.

3. The medical equipment mounting system of claim 2, wherein the first support wall includes a coupling member having an alignment tab configured to be received within an alignment slot of the second support wall.

4. The medical equipment mounting system of claim 3, wherein the coupling member further includes a lower extension configured to facilitate alignment of the plurality of different types of mounting plates.

5. The medical equipment mounting system of claim 1, wherein the one or more features include plate alignment cutouts formed in the plurality of support members, the plate alignment cutouts being sized and shaped to receive portions of the plurality of different types of mounting plates.

6. The medical equipment mounting system of claim 1, wherein the opposing walls include extended portions configured to receive wall ends of adjacent opposing walls when the frame is assembled.

7. The medical equipment mounting system of claim 1, further comprising a supply air array having a plurality of openings, wherein the mount is coupled to the supply air array along a perimeter thereof.

8. A mount for medical equipment, comprising:
a frame having opposing walls that define a perimeter;
a plurality of support walls positioned at corner regions of the frame, each support wall including a coupling member having an alignment tab configured to engage with an alignment slot of an adjacent support wall; and
a plurality of cutouts formed in the support walls, the cutouts configured to receive and align different mounting plates to the mount.

9. The mount of claim 8, wherein the coupling member further includes a lower extension that extends below a bottom surface of the support wall.

10. The mount of claim 9, wherein the lower extension is configured to facilitate alignment of the different mounting plates by providing a positioning reference.

11. The mount of claim 8, wherein the plurality of cutouts include plate alignment cutouts formed at bottom portions of the support walls, the plate alignment cutouts being sized to receive edge portions of the different mounting plates.

12. The mount of claim 11, wherein the plurality of cutouts further include support wall cutouts that define engagement portions configured to abut against interior portions of the opposing walls.

13. The mount of claim 8, wherein the opposing walls include extended portions at ends thereof, the extended portions configured to receive wall ends of adjacent opposing walls when the frame is assembled.

14. The mount of claim 8, wherein each support wall includes mounting openings configured to receive fasteners for securing the different mounting plates to the mount.

15. A medical equipment mounting system for an operating room, comprising:
a supply air array having a frame with a plurality of openings; and
an adaptive mount coupled to the supply air array, the adaptive mount having a plurality of support members at corner regions, wherein the support members include alignment features configured to accommodate mounting plates from different manufacturers.

16. The medical equipment mounting system of claim 15, wherein each of the plurality of support members comprises a first support wall and a second support wall arranged in a perpendicular configuration at each corner region.

17. The medical equipment mounting system of claim 16, wherein the first support wall includes a coupling member having an alignment tab configured to be received within an alignment slot of the second support wall.

18. The medical equipment mounting system of claim 17, wherein the coupling member further includes a lower extension that extends below a bottom surface of the first support wall to facilitate positioning of the mounting plates.

19. The medical equipment mounting system of claim 15, wherein the alignment features include plate alignment cutouts formed in the support members, the plate alignment cutouts being sized and shaped to receive edge portions of mounting plates having different dimensions.

20. The medical equipment mounting system of claim 19, wherein the support members further include support wall cutouts that define engagement portions configured to abut against interior portions of the supply air array frame when the adaptive mount is coupled thereto.
